(19) United States
(12) Patent Application Publication
(10) Pub. No.: US 2024/0262721 A1
(43) Pub. Date: Aug. 8, 2024

(54) ELECTROLYZED WATER SPRAY DEVICE

(71) Applicant: Panasonic Intellectual Property Management Co., Ltd., Kadoma-shi, Osaka (JP)

(72) Inventors: Yuji TAKASE, Aichi (JP); Fumiaki ASAI, Aichi (JP); Shin CHIBA, Aichi (JP); Mai ISHIGURO, Aichi (JP); Yumiko KANIE, Aichi (JP)

(21) Appl. No.: 18/567,055

(22) PCT Filed: Jun. 1, 2022
(86) PCT No.: PCT/JP2022/022258

§ 371 Date: Dec. 5, 2023

(30) Foreign Application Priority Data
Jun. 14, 2021 (JP) 2021-098388

(51) Int. Cl. C02F 1/461; A61L 9/14

(52) U.S. Cl. CPC C02F 1/4618; A61L 9/145; A61L 2209/111; A61L 2209/213; C02F 2001/46185; C02F 2201/4615; C02F 2201/4618; C02F 2209/42; C02F 2303/14

(57) ABSTRACT

An electrolyzed water spraying device of the present disclosure includes: a short-term maintenance water storage portion (30) that stores liquid and requires short-term maintenance; a long-term maintenance water storage portion (31) that stores liquid and requires long-term maintenance; and a notification controller (43) that notifies each of the timing of the next short-term maintenance and the timing of the next long-term maintenance. The notification controller (43) simultaneously performs the notification of the timing of the next short-term maintenance and the notification of the timing of the next long-term maintenance when the timing of the next short-term maintenance and the timing of the next long-term maintenance are close to each other.

### TECHNICAL FIELD

The present disclosure relates to an electrolyzed water spraying device.

### BACKGROUND ART

An electrolyzed water spraying device that generates and discharges electrolyzed water containing hypochlorous acid by electrolysis in order to remove (including inactivation of) bacteria, fungi, viruses, odors, and the like in the air is known (see, for example, Patent Literature 1). Generating hypochlorous acid requires charging an electrolytic accelerator such as a salt into water and generating water containing chloride ions.

### CITATION LIST

### Patent Literature

PTL 1: Unexamined Japanese Patent Publication No. 2019-24811

### SUMMARY OF THE INVENTION

In a conventional electrolyzed water spraying device, electrolyzed water is generated in a water storage portion, the generated electrolyzed water and air sucked from the outside are continuously brought into contact with each other inside, and the air after the contact is directly discharged from the water storage portion to the outside. Whereas, when the electrolyzed water storage portion that generates and stores the electrolyzed water and a spray water storage portion that stores the electrolyzed water supplied from the electrolyzed water storage portion and discharged to the outside are separated from each other, the times when the maintenance is required are different between the electrolyzed water storage portion and the spray water storage portion.

Specifically, continuous electrolysis in the electrolyzed water storage portion causes inorganic salts of calcium carbonate, calcium sulfate, silica, and the like contained in water to adhere as impurities to an electrolysis unit performing electrolysis, which may shorten the life of the electrolysis unit. When the electrolyzed water is supplied to the spray water storage portion, most of the impurities are sent together with the electrolyzed water, but some of the impurities remain in the electrolyzed water storage portion. Therefore, it is necessary to periodically perform maintenance such as cleaning of the electrolyzed water storage portion although the frequency is not high.

When the spray water storage portion receives the electrolyzed water from the electrolyzed water storage portion, impurities are mixed in the electrolyzed water. Therefore, the impurities accumulate in the spray water storage portion. Accumulation of the impurities in the spray water storage portion deteriorates the performance of a filter for discharging the electrolyzed water. Therefore, it is necessary to periodically perform maintenance such as cleaning of the spray water storage portion.

The conventional electrolyzed water spraying device needs to have two types of notification functions, one notifying a user that maintenance of the electrolyzed water storage portion is necessary, the other notifying the user that maintenance of the spray water storage portion is necessary. If the respective notifications are independent in this case, immediately after the user has completed one maintenance, a notification that prompts the user to carry out the other maintenance may be made, which is troublesome and less convenient for the user.

An object of the present disclosure is to provide a technique for improving the convenience of the user regarding maintenance.

An electrolyzed water spraying device according to the present disclosure is an electrolyzed water spraying device that discharges electrolyzed water, and includes: a short-term maintenance water storage portion that stores liquid and requires short-term maintenance that is maintenance on a short-term basis; a long-term maintenance water storage portion that stores the liquid and requires long-term maintenance that is maintenance on a long-term basis longer than the maintenance of the short-term maintenance water storage portion; and a notification controller that notifies each of a timing of a next short-term maintenance of the short-term maintenance water storage portion and a timing of a next long-term maintenance of the long-term maintenance water storage portion. The notification controller simultaneously performs a notification of the next short-term maintenance and a notification of the next long-term maintenance when the timing of the next short-term maintenance and the timing of the next long-term maintenance are close to each other.

According to the present disclosure, the convenience of the user regarding the maintenance can be improved.

### BRIEF DESCRIPTION OF THE DRAWINGS

FIG. 1 is a perspective view of an electrolyzed water spraying device according to a first exemplary embodiment of the present disclosure.

FIG. 2 is a sectional view of the electrolyzed water spraying device according to the first exemplary embodiment of the present disclosure as viewed from a right side surface.

FIG. 3 is a schematic functional block diagram of a controller and peripheral units according to the first exemplary embodiment of the present disclosure.

FIG. 4 is a flowchart illustrating a control procedure by the controller according to the first exemplary embodiment of the present disclosure.

FIG. 5 is a diagram illustrating an example in which the control procedure by the controller according to the first exemplary embodiment of the present disclosure is represented in time series.

FIG. 6 is a flowchart illustrating a control procedure by a controller according to a second exemplary embodiment of the present disclosure.

FIG. 7 is a diagram illustrating an example in which the control procedure by the controller according to the second exemplary embodiment of the present disclosure is represented in time series.

### DESCRIPTION OF EMBODIMENT

Exemplary embodiments of the present disclosure will now be explained with reference to the drawings. It is to be noted, however, that the exemplary embodiments described below are illustrations each embodying a technical idea of the present invention, and the present disclosure is not specifically limited to the following exemplary embodiments. In particular, materials, shapes, constituent elements, disposition and relative disposition of the constituent elements, and the like described in the exemplary embodiments are merely examples, and the scope of the present disclosure is not intended to be limited to them. Furthermore, in the drawings, substantially the same components are denoted by the same reference marks, and redundant descriptions thereof will be omitted or simplified.

A conventional electrolyzed water spraying device dissolves an electrolytic accelerator in water in a water storage portion to generate water containing chloride ions, and then electrolyzes the water containing chloride ions to generate electrolyzed water containing active oxygen species. In the conventional electrolyzed water spraying device, in the water storage portion, the generated electrolyzed water and the air sucked from the outside are continuously brought into contact with each other inside, and the air after the contact is discharged to the outside by rotation of the fan. Therefore, the electrolyzed water in the water storage portion is easily contaminated by contact with air. Contamination of the electrolyzed water may deteriorate the electrolysis unit. Therefore, in the present exemplary embodiment, an electrolyzed water spraying device having a new configuration with which an electrolysis unit is hardly deteriorated will be described.

### First Exemplary Embodiment

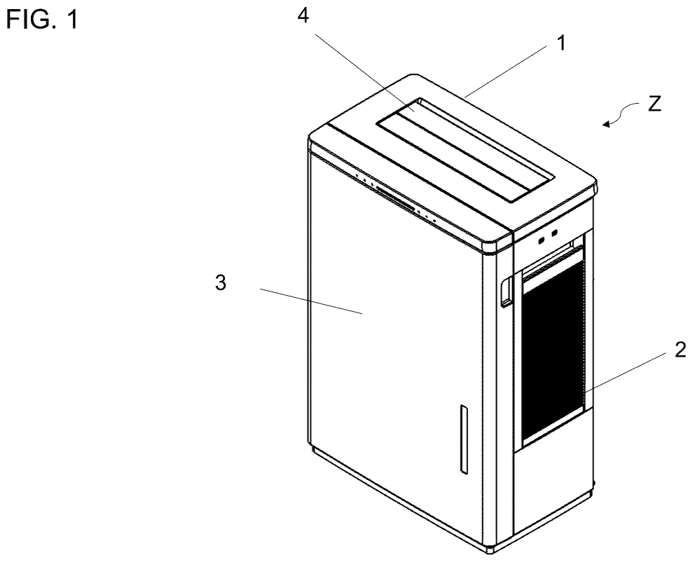

An exemplary embodiment of the present disclosure will now be explained with reference to the accompanying drawings. FIG. 1 is a perspective view of electrolyzed water spraying device Z according to a first exemplary embodiment of the present disclosure. As illustrated in FIG. 1, electrolyzed water spraying device Z is a device that discharges electrolyzed water, and includes body case 1.

Body case 1 is a substantially box-shaped box body, and includes air inlets 2, air outlet 4, and panel 3. In a state where electrolyzed water spraying device Z in FIG. 1 is installed, a surface on a side where panel 3 is provided is referred to as “front surface”, a surface facing the front surface of electrolyzed water spraying device Z is referred to as “back surface”, and a side surface on a right side as viewed from the front surface side of electrolyzed water spraying device Z is referred to as “right side surface”.

Air inlets 2 are grid-like openings that are provided on both side surfaces of body case 1 and take air outside body case 1 into body case 1.

Air outlet 4 is an openable and closable opening that is provided on the back side of the top surface of body case 1 and blows out the air taken into body case 1 from air inlets 2 to the outside of body case 1. In FIG. 1, air outlet 4 is closed surface.

Panel 3 is provided on a front surface of body case 1. Panel 3 is an openable and closable cover, and is mainly formed of plastic resin.

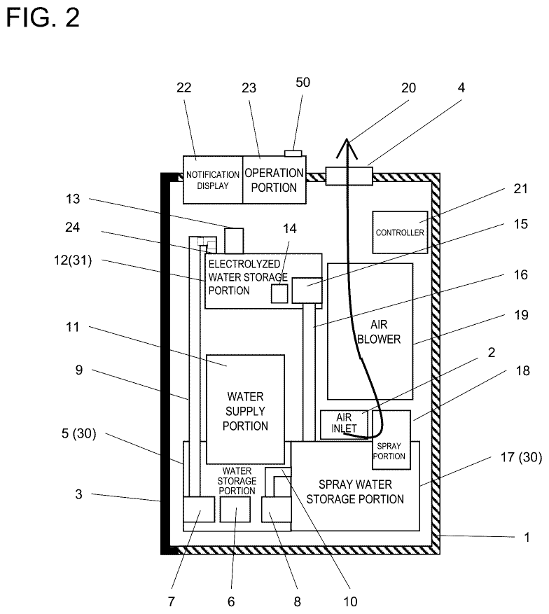

FIG. 2 is a sectional view of electrolyzed water spraying device Z in FIG. 1 as viewed from a right side surface. FIG. 2 illustrates an internal configuration of electrolyzed water spraying device Z.

As illustrated in FIG. 2, body case 1 includes water storage portion 5, first pump 7, first water conveyance path 9, second pump 8, second water conveyance path 10, water supply portion 11, electrolyzed water storage portion 12, electrolytic accelerator charge portion 13, electrolysis unit 14, third pump 15, third water conveyance path 16, spray water storage portion 17, spray portion 18, air blower 19, air passage 20, and controller 21.

Water storage portion 5 has a box shape with a top surface opened, has a structure that can store water, and stores water supplied from water supply portion 11 described later. For example, water storage portion 5 is disposed in a lower portion of electrolyzed water spraying device Z. Water storage portion 5 is integrated with spray water storage portion 17 described later, and is detachable together with spray water storage portion 17 when water storage portion 5 is slid in the front-rear direction with respect to body case 1 after panel 3 is opened. The water storage portion 5 has a structure that can hold water supply portion 11. Water storage portion 5 includes detector 6.

Detector 6 detects a water level in water storage portion 5. For example, detector 6 includes a magnet equipped with a float having buoyancy inside, and a magnetic force detector that is located at a position facing the magnet equipped with a float and detects the magnetic force of the magnet. However, this configuration need not be adopted as long as the water level can be detected. Detector 6 detects water shortage in water storage portion 5 by detecting that the water level in water storage portion 5 becomes equal to or lower than the water-shortage water level. The water-shortage water level is, for example, a water level lower than the opening and closing portion of water supply portion 11, and is a water level that causes a problem when water in water storage portion 5 is supplied from first pump 7 and second pump 8 described later. The water level that causes a problem when water in water storage portion 5 is supplied from first pump 7 and second pump 8 is a water level at which air is mixed into first pump 7 and second pump 8 when water in water storage portion 5 is supplied from first pump 7 and second pump 8. This is because decrease in the water level causes air to enter the pump, leading to a decrease in the amount of supplied water and to generation of abnormal noise. When the water level in water storage portion 5 becomes equal to or lower than the water-shortage water level, detector 6 transmits a water-shortage detection signal to controller 21.

First pump 7 is disposed in water storage portion 5, and is connected to first water conveyance path 9. First pump 7 operates in accordance with an instruction from controller 21 to pump the water stored in water storage portion 5 toward first water conveyance path 9. First water conveyance path 9 is a pipe connecting water storage portion 5 and electrolyzed water storage portion 12, and has supply port 24 at an end on electrolyzed water storage portion 12 side. Water pumped up by first pump 7 flows in first water conveyance path 9, and is supplied from supply port 24 to electrolyzed water storage portion 12. That is, first pump 7 and first water conveyance path 9 supply water from water storage portion 5 to electrolyzed water storage portion 12.

Second pump 8 is disposed in water storage portion 5, and is connected to second water conveyance path 10. Second pump 8 operates in accordance with an instruction from controller 21 to pump the water stored in water storage portion 5 toward second water conveyance path 10. Second water conveyance path 10 is a pipe connecting water storage portion 5 and spray water storage portion 17. Water pumped up by second pump 8 flows in second water conveyance path 10, and is supplied to spray water storage portion 17. That is, second pump 8 and second water conveyance path 10 supply water from water storage portion 5 to spray water storage portion 17.

Water supply portion 11 is disposed above water storage portion 5. Water supply portion 11 is a tank that stores water therein, and has a structure detachable from water storage portion 5. Water supply portion 11 can be taken out in a state where panel 3 is opened. A lid (not illustrated) is provided at an opening (not illustrated) of water supply portion 11, and an opening and closing portion (not illustrated) is provided at the center of the lid. When the opening and closing portion is opened, the water in water supply portion 11 is supplied to water storage portion 5.

Specifically, water supply portion 11 is attached to water storage portion 5 with the opening of water supply portion 11 faced downward, thereby opening the opening and closing portion. That is, when water supply portion 11 storing water is attached to water storage portion 5, the opening and closing portion is opened to supply water to water storage portion 5, and water is stored in water storage portion 5. When the water level in water storage portion 5 rises and reaches the lid, the water supply is stopped because the opening of water supply portion 11 is water-sealed. When water remains in water supply portion 11, water in water supply portion 11 is supplied to water storage portion 5 every time the water level in water storage portion 5 lowers. As a result, the water level in water storage portion 5 is kept constant.

Electrolyzed water storage portion 12 has a box shape with a top surface opened, and is disposed below supply port 24 at an end of first water conveyance path 9 on a side of electrolyzed water storage portion 12. Electrolyzed water storage portion 12 stores water supplied from supply port 24. On the upper side of electrolyzed water storage portion 12, electrolytic accelerator charge portion 13 is disposed side by side with supply port 24.

Electrolytic accelerator charge portion 13 can be loaded with an electrolytic accelerator in the portion, and rotates a tablet charge member (not illustrated) when receiving an instruction to charge the electrolytic accelerator from controller 21. When the tablet charge member rotates, the electrolytic accelerator drops into electrolyzed water storage portion 12. Electrolytic accelerator charge portion 13 counts the number of electrolytic accelerators dropped into electrolyzed water storage portion 12, and stops the rotation of the tablet charge member when determining that one tablet of the electrolytic accelerator has dropped into electrolyzed water storage portion 12. That is, electrolytic accelerator charge portion 13 charges an electrolytic accelerator into electrolyzed water storage portion 12. When electrolytic accelerator dissolves into water in electrolyzed water storage portion 12, water containing chloride ions is generated in electrolyzed water storage portion 12. One example of the electrolytic accelerator is sodium chloride, and is formed as an electrolysis acceleration tablet.

Electrolysis unit 14 includes electrodes, and is set to immerse the electrodes in water in electrolyzed water storage portion 12. Electrolysis unit 14 electrochemically electrolyzes water containing chloride ions in electrolyzed water storage portion 12 by energizing the electrodes to generate electrolyzed water containing active oxygen species. Herein, the active oxygen species are oxygen molecules having an oxidation activity higher than the oxidation activity of normal oxygen and substances related with them. For example, active oxygen species include so-called narrow sense of active oxygen such as superoxide anion, singlet oxygen, hydroxyl radical, or hydrogen peroxide, and so-called broad sense of active oxygen such as ozone or hypochlorous acid (hypohalous acid).

Electrolysis unit 14 generates electrolyzed water by repeating one cycle a plurality of times, where the one cycle is an energization time for performing energization for electrolysis and a time after stop of the energization, that is, a non-energization time, which is a time when the energization is not performed. By providing the non-energization time for electrolysis unit 14, the life of electrolysis unit 14 is extended. When the energization time is elongated with respect to the non-energization time, electrolyzed water containing a larger amount of active oxygen species per cycle is generated. When the non-energization time is elongated with respect to the energization time, generation of active oxygen species per cycle can be suppressed. Further, when the amount of power during the energization time is increased, electrolyzed water containing a larger amount of active oxygen species is generated. As described above, it can be said that electrolyzed water storage portion 12 is a tank for generating electrolyzed water from water into which an electrolytic accelerator is charged and for storing the electrolyzed water.

Third pump 15 is disposed in electrolyzed water storage portion 12, and is connected to third water conveyance path 16. Third pump 15 operates in accordance with an instruction from controller 21, and pumps electrolyzed water stored in electrolyzed water storage portion 12 toward third water conveyance path 16. Third water conveyance path 16 is a pipe connecting electrolyzed water storage portion 12 and spray water storage portion 17. Water pumped by third pump 15 flows in third water conveyance path 16, and is supplied to spray water storage portion 17. That is, third pump 15 and third water conveyance path 16 supply the electrolyzed water from electrolyzed water storage portion 12 to spray water storage portion 17.

Spray water storage portion 17 has a box shape with a top surface opened, and stores spray water obtained by mixing water supplied from water storage portion 5 and electrolyzed water supplied from electrolyzed water storage portion 12. This corresponds to diluting the electrolyzed water supplied from electrolyzed water storage portion 12 with the water supplied from water storage portion 5.

Spray portion 18 is a filter having water retainability and having a cylindrical shape, and has a configuration in which a hole through which air can flow is provided in a circumferential portion. Spray portion 18 is mounted on spray water storage portion 17 so that one end of spray portion 18 immerses in spray water storage portion 17, having a structure that causes electrolyzed water and air are continuously brought into contact with each other when spray portion 18 is rotated by a drive unit (not illustrated) provided in spray water storage portion 17.

Air blower 19 is provided at the center of body case 1, and includes, for example, a motor (not illustrated), a sirocco fan (not illustrates), and a casing (not illustrated) surrounding them. Air blower 19 takes in air outside body case 1 from air inlets 2 by rotating of a sirocco fan by a motor. The air taken in from air inlets 2 passes through spray portion 18 and is taken in a casing from the lower portion of the casing. The air taken into the casing is discharged from air outlet 4 to the outside of body case 1. As a result, the air taken in from air inlets 2 is discharged from air outlet 4.

Air passage 20 allows air inlets 2 and air outlet 4 to communicate with each other. On air passage 20, air inlets 2, spray portion 18, air blower 19, and air outlet 4 are provided in this order. When the sirocco fan of air blower 19 rotates, the air outside body case 1 entering air passage 20 from air inlets 2 is sequentially discharged to the outside of body case 1 through spray portion 18, air blower 19, and air outlet 4. In this case, the air taken in passes through spray portion 18, so that the electrolyzed water having permeated spray portion 18 is discharged to the outside of body case 1. Electrolyzed water spraying device Z does not necessarily spray electrolyzed water itself, and spraying electrolyzed water includes also releasing active oxygen species derived from (including volatilization of) the resultant electrolyzed water.

Controller 21 controls electrolyzed water spraying device Z, and details of the control content will be described later.

The top surface of body case 1 is provided with notification display 22 and operation portion 23.

Notification display 22 is for prompting the user to perform maintenance work. Notification display 22 is, for example, a light emitting diode (LED), and prompts the user to perform maintenance work, by turning on the LED in accordance with an instruction from controller 21. The LED includes a short-term maintenance LED to notify short-term maintenance and a long-term maintenance LED to notify long-term maintenance.

Operation portion 23 includes a button operable by the user, and includes at least maintenance completion button 50. When the maintenance work is completed by the user and maintenance completion button 50 is pressed, the statement that maintenance completion button 50 has been pressed is transmitted to controller 21.

In the present exemplary embodiment, as the maintenance performed by a user, there are short-term maintenance and long-term maintenance. The members requiring the short-term maintenance are spray water storage portion 17 and water storage portion 5. That is, it can also be said that spray water storage portion 17 and water storage portion 5 are short-term maintenance water storage portion 30 storing the liquid and requiring the short-term maintenance that is maintenance on a short-term basis. When the electrolyzed water is supplied from electrolyzed water storage portion 12, in spray water storage portion 17, because impurities that are inorganic salts of calcium carbonate, calcium sulfate, silica, and the like have been mixed in the electrolyzed water, the impurities accumulate in spray water storage portion 17. As the impurities accumulate, the performance of the filter of spray portion 18 deteriorates. Therefore, it is necessary to periodically perform at least one of draining or cleaning. Water storage portion 5 is integrated with spray water storage portion 17, and thus water storage portion 5 and spray water storage portion 17 are simultaneously subjected to at least one of draining or cleaning. Herein, the draining is draining from short-term maintenance water storage portion 30 (spray water storage portion 17 and water storage portion 5). The cleaning is, for example, washing of short-term maintenance water storage portion 30 with water. After performing of at least one of draining or cleaning, a user needs to perform water supply maintenance.

The water supply maintenance is water supply to short-term maintenance water storage portion 30. This corresponds to replenishment of new water to water supply portion 11 and attachment of water supply portion 11 to water storage portion 5. That is, the short-term maintenance includes at least one of draining or cleaning of short-term maintenance water storage portion 30 and water supply maintenance after at least one of the draining or the cleaning is performed. In addition, a filter of spray portion 18 may be included as a member requiring short-term maintenance.

A member requiring long-term maintenance in the present exemplary embodiment is electrolyzed water storage portion 12. That is, it can also be said that electrolyzed water storage portion 12 is long-term maintenance water storage portion 31 storing liquid and requiring the long-term maintenance that is maintenance on a long-term basis longer than the maintenance of short-term maintenance water storage portion 30. In electrolyzed water storage portion 12, continuing electrolysis causes inorganic salts of calcium carbonate, calcium sulfate, silica, and the like contained in water to adhere as impurities to electrolysis unit 14 that performs electrolysis, which may shorten the life of electrolysis unit 14. When the electrolyzed water is sent to spray water storage portion 17, most of the impurities are sent together with the electrolyzed water, but some of the impurities remain in electrolyzed water storage portion 12. Therefore, it is necessary to periodically perform at least one of draining or cleaning although the frequency is not high as compared to the conventional electrolyzed water spraying device. Herein, the draining is draining of long-term maintenance water storage portion 31 (electrolyzed water storage portion 12), and the cleaning is, for example, washing of long-term maintenance water storage portion 31 with water. That is, the long-term maintenance includes at least one of draining or cleaning of long-term maintenance water storage portion 31. The determination condition for determining that the short-term maintenance is necessary and the determination condition for determining that the long-term maintenance is necessary will be described later.

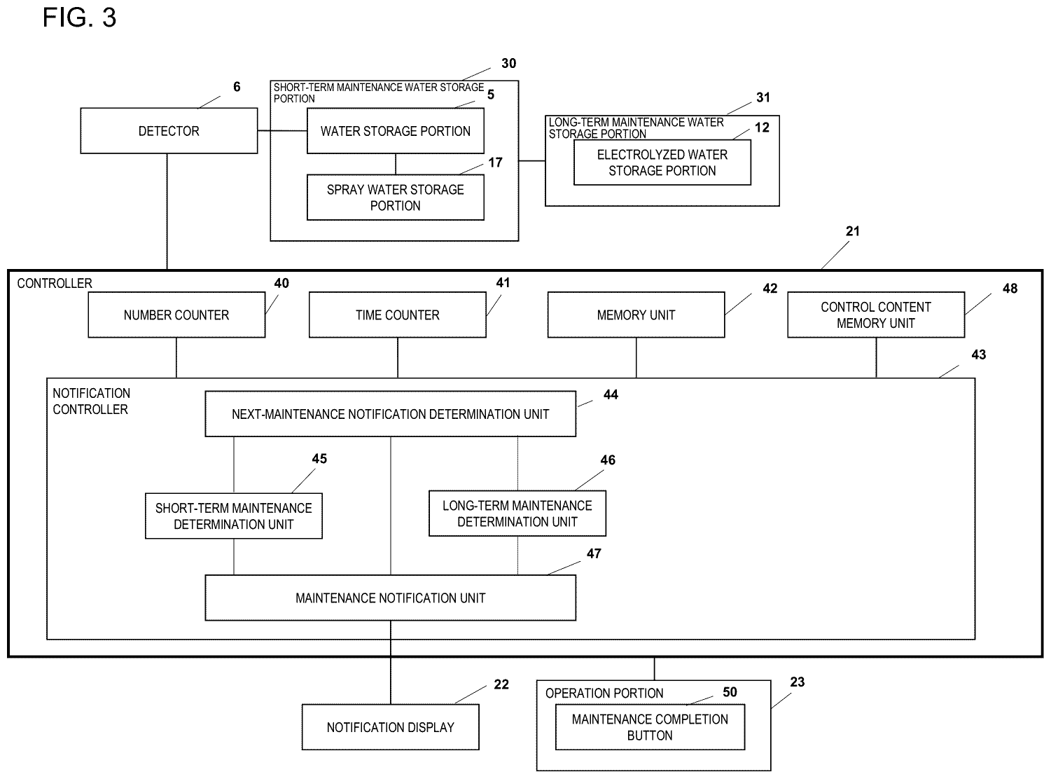

Then, each function of controller 21 according to the exemplary embodiment of the present disclosure will be described with reference to FIG. 3. FIG. 3 is a schematic functional block diagram of controller 21 and peripheral units. Controller 21 includes number counter 40, time counter 41, memory unit 42, and notification controller 43.

Number counter 40 counts the number of times of water shortage in water storage portion 5 detected by detector 6 as the number of times of detection, water storage portion 5 being short-term maintenance water storage portion 30. In counting the number of times of detection, when the water-shortage detection signal is received from detector 6, the number of times of detection is increased by one. In addition, instead of the number of times of detection of the water-shortage water level, the number of times of supplying water may be counted as the number of times of detection. Counting the number of times of supplying water is performed, for example, when water supply maintenance is performed by a user in a state where the water level in water storage portion 5 is equal to or less than the water-shortage water level. That is, when the water supply maintenance is performed, the water level in water storage portion 5 exceeds the water-shortage water level and then detector 6 transmits the water supply detection signal, and controller 21 receives the water supply detection signal and then the number of times of supplying water is increased by one.

Time counter 41 counts the elapsed time from completion of the previous long-term maintenance for long-term maintenance water storage portion 31. Hereinafter, the elapsed time from completion of the previous long-term maintenance is referred to as “long-term maintenance elapsed time”, and the elapsed time from completion of the previous short-term maintenance is referred to as “short-term maintenance elapsed time”. The completion of maintenance is determined when maintenance completion button 50 of operation portion 23 is pressed and controller 21 receives a signal from maintenance completion button 50.

Memory unit 42 stores time threshold value T1 or detection number threshold value K1 used for determination of a notification of short-term maintenance, and time threshold value T2 larger than time threshold value T1 and used for determination of a notification of long-term maintenance.

Time threshold value T1 is a time for determining whether or not the maintenance of short-term maintenance water storage portion 30 is necessary due to accumulation of the impurities in spray water storage portion 17 that is short-term maintenance water storage portion 30, and is, for example, a value determined previously by an experiment or the like, and can be optionally set. In the present exemplary embodiment, as an example, the value is set to 30 days.

Number threshold value K1 is a number of times of detection for determining whether or not the maintenance of short-term maintenance water storage portion 30 is necessary due to accumulation of the impurities in spray water storage portion 17 that is short-term maintenance water storage portion 30, and is, for example, a value determined previously by an experiment or the like, and can be optionally set. In the present exemplary embodiment, as an example, the value is set to 30. In the present exemplary embodiment, water-shortage detection is assumed to be performed about once a day. Therefore, the time to reach number threshold value K1 is assumed to be about 30 days close to time threshold value T1. However, the consumption amount of electrolyzed water changes depending on various factors such as seasons and weather, and thus the time from the previous water shortage detection to the next water shortage detection differs every time, and the time until the number of times of detection reaches number threshold value K1 is not necessarily 30 days. For example, when a large amount of electrolyzed water is consumed for sterilization and deodorization for a long period, the number of times of detection may reach number threshold value K1 in 25 days. The amount of impurities accumulated in spray water storage portion 17 is constant until the number of times of detection reaches number threshold value K1, and thus it is possible to accurately determine whether or not the maintenance of short-term maintenance water storage portion 30 is required, using number threshold value K1.

Time threshold value T2 is a time for determining whether or not the maintenance of long-term maintenance water storage portion 31 is necessary due to accumulation of the impurities in electrolyzed water storage portion 12 that is long-term maintenance water storage portion 31, and is, for example, a value determined previously by an experiment or the like, and can be optionally set. In the present exemplary embodiment, as an example, the value is set to 365 days. Impurities are less likely to accumulate in electrolyzed water storage portion 12 as compared to spray water storage portion 17, and thus time threshold value T2 for determining whether or not maintenance is necessary is set to be larger than time threshold value T1.

Notification controller 43 notifies the maintenance timings of short-term maintenance water storage portion 30 and long-term maintenance water storage portion 31. Notification controller 43 includes next-maintenance notification determination unit 44, short-term maintenance determination unit 45, long-term maintenance determination unit 46, and maintenance notification unit 47.

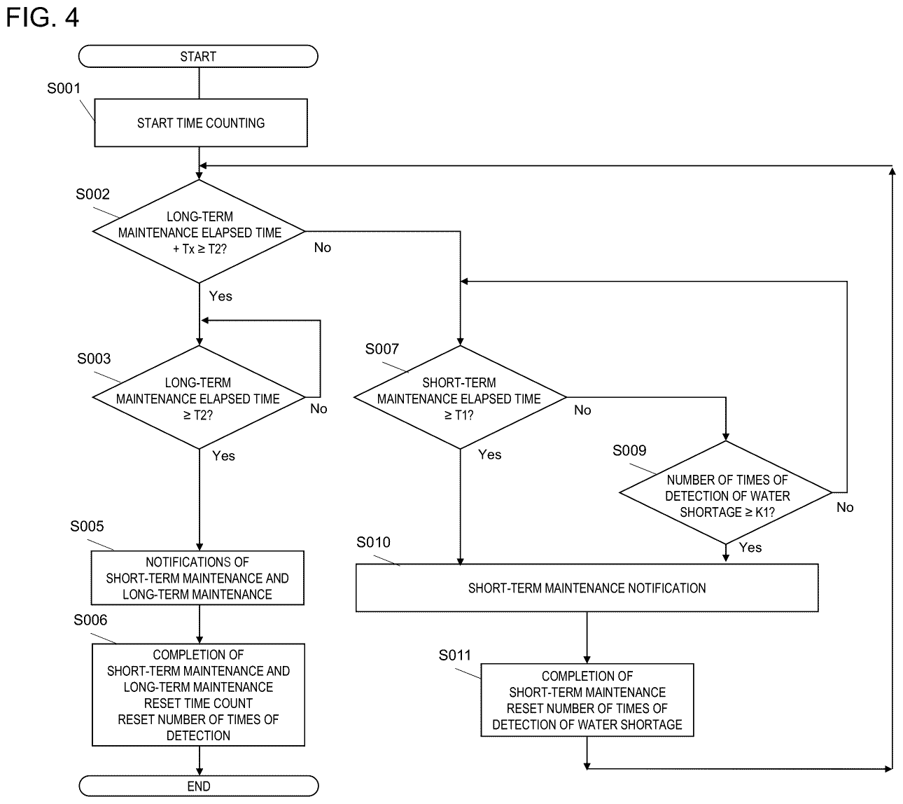

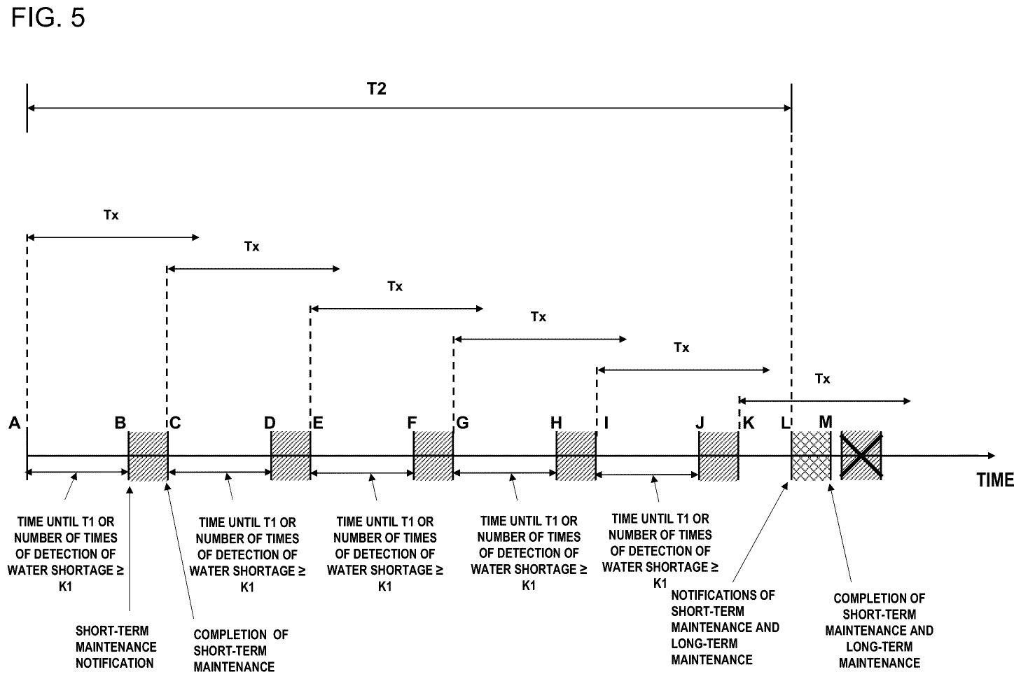

Next-maintenance notification determination unit 44 determines whether or not the timing of the next short-term maintenance and the timing of the next long-term maintenance are close to each other. Details of the determination method will be described later with reference to FIGS. 4 and 5. When the timing of the next short-term maintenance and the timing of the next long-term maintenance are close to each other, next-maintenance notification determination unit 44 transmits, to maintenance notification unit 47, the statement that the notification of the short-term maintenance and the notification of the long-term maintenance are performed simultaneously in the next maintenance notification. When the timing of the next short-term maintenance and the timing of the next long-term maintenance are not close to each other, next-maintenance notification determination unit 44 transmits, to maintenance notification unit 47, the statement that the notification of the short-term maintenance is performed in the next maintenance notification.

Short-term maintenance determination unit 45 determines whether or not notification of short-term maintenance is necessary. Short-term maintenance determination unit 45 makes a determination based on at least one of the number of times of water shortage in water storage portion 5 that is short-term maintenance water storage portion 30 or the number of times of water supply maintenance that is water supply to water storage portion 5 that is short-term maintenance water storage portion 30, and the elapsed time from the previous short-term maintenance for short-term maintenance water storage portion 30. In other words, the determination may be made based on both the number of times of water shortage or the number of times of water supply maintenance and the elapsed time from the previous short-term maintenance. In the present exemplary embodiment, as an example, short-term maintenance determination unit 45 makes a determination based on the number of times of water shortage in water storage portion 5 that is short-term maintenance water storage portion 30 and the elapsed time from the previous short-term maintenance for short-term maintenance water storage portion 30.

Specifically, short-term maintenance determination unit 45 determines that the notification of the short-term maintenance is necessary when the elapsed time from completion of the previous short-term maintenance is equal to or more than time threshold value T1 or the number of times of detection of water shortage from completion of the previous short-term maintenance is equal to or more than number threshold value K1. When determining that the notification of the short-term maintenance is necessary, short-term maintenance determination unit 45 transmits, to maintenance notification unit 47, the statement that the notification is necessary.

Only when next-maintenance notification determination unit 44 determines that the notification of the short-term maintenance and the notification of the long-term maintenance are to be performed simultaneously in the next maintenance notification, long-term maintenance determination unit 46 may determine whether or not the notification of the next short-term maintenance is necessary. In this case, whether or not the notification of the short-term maintenance (and the notification of the long-term maintenance) is necessary is determined based on the elapsed time from the previous long-term maintenance for long-term maintenance water storage portion 31, and the details will be described later.

Long-term maintenance determination unit 46 determines whether or not notification of long-term maintenance is necessary. Long-term maintenance determination unit 46 makes a determination based on the elapsed time from the previous long-term maintenance for long-term maintenance water storage portion 31. Specifically, long-term maintenance determination unit 46 determines that notification of long-term maintenance is necessary when the count time of time counter 41 is equal to or more than time threshold value T2. When determining that the notification of the long-term maintenance is necessary, long-term maintenance determination unit 46 transmits, to maintenance notification unit 47, the statement that the notification is necessary.

When next-maintenance notification determination unit 44 determines that the notification of the short-term maintenance and the notification of the long-term maintenance are to be performed simultaneously in the next maintenance notification, short-term maintenance determination unit 45 may determine whether or not the notification of the next long-term maintenance is necessary. In this case, whether or not the notification of the long-term maintenance (and the notification of the short-term maintenance) is necessary is determined based on at least one of the number of times of water shortage in water storage portion 5 that is short-term maintenance water storage portion 30 or the number of times of water supply maintenance that is water supply to water storage portion 5 that is short-term maintenance water storage portion 30 and the elapsed time from the previous short-term maintenance for short-term maintenance water storage portion 30.

Maintenance notification unit 47 receives a signal from next-maintenance notification determination unit 44, and a signal from short-term maintenance determination unit 45 or long-term maintenance determination unit 46. When maintenance notification unit 47 receives, from next-maintenance notification determination unit 44, a statement that the notification of the short-term maintenance and the notification of the long-term maintenance are performed simultaneously in the next maintenance notification, and further receives, from short-term maintenance determination unit 45 or long-term maintenance determination unit 46, a determination that the notification is necessary, the maintenance notification unit 47 simultaneously turns on the short-term maintenance LED and the long-term maintenance LED in notification display 22 to perform the notification of the short-term maintenance and the notification of the long-term maintenance simultaneously.

When maintenance notification unit 47 receives, from next-maintenance notification determination unit 44, the statement that the notification of the short-term maintenance is to be performed in the next maintenance notification, and further receives, from short-term maintenance determination unit 45, a determination that the notification is necessary, maintenance notification unit 47 turns on the short-term maintenance LED in notification display 22 to perform the notification of the short-term maintenance.

Herein, controller 21 is configured by a computer. This computer executes a program, and thus, functions of the subject of devices or methods in the present disclosure are realized. The computer includes, as a main hardware configuration, a processor that acts according to the program. The type of the processor is not limited as long as the processor can implement the function by executing the program. The processor includes one or a plurality of electronic circuits including a semiconductor integrated circuit or a large scale integration (LSI). The plurality of electronic circuits may be integrated on one chip or may be provided on a plurality of chips. The plurality of chips may be aggregated into one device or may be provided in a plurality of devices. The program is recorded in a computer-readable non-transitory recording medium such as a read only memory (ROM). The program may be stored previously in the recording medium, or may be provided to the recording medium via a wide area communication network including the Internet and the like.

An operation of controller 21 with the above configuration will be described with reference to FIGS. 4 and 5. FIG. 4 is a flowchart illustrating a control procedure by controller 21. Herein, in the flowchart, numbers with S as an initial letter are assigned. For example, S001 or the like indicates a processing step. The magnitude of the numerical value indicating the processing step has no relation to the processing order. FIG. 5 is a diagram illustrating an example of the control procedure illustrated in FIG. 4 in time series.

In the present exemplary embodiment, the operation of controller 21 after completion of both the previous long-term maintenance and the short-term maintenance will be described. First, time counter 41 starts counting the elapsed time from completion of the previous long-term maintenance (S001). In FIG. 5, counting is started at predetermined time A (timing A).

Next-maintenance notification determination unit 44 determines whether or not the timing of the next short-term maintenance and the timing of the next long-term maintenance are close to each other. Specifically, at the time of completion of the short-term maintenance, next-maintenance notification determination unit 44 determines whether or not the time obtained by adding predetermined time Tx to the long-term maintenance elapsed time counted by time counter 41 is equal to or more than time threshold value T2 (S002).

Predetermined time Tx is used to determine whether to perform the notification of the short-term maintenance or to perform both the notification of the short-term maintenance and the notification of the long-term maintenance in the next maintenance notification. For example, when predetermined time Tx is set to be a numerical value close to time threshold value T1 used for determination of the short-term maintenance, it is possible to determine whether or not the timing of the next short-term maintenance and the timing of the next long-term maintenance are close to each other. To determine whether or not the timing of the next short-term maintenance and the timing of the next long-term maintenance are close to each other, predetermined time Tx is desirably set to be equal to or less than twice time threshold value T1. Setting predetermined time Tx to be larger than twice time threshold value T1 causes the period from the previous short-term maintenance to the next short-term maintenance to be excessively long. For example, the vacant period of two short-term maintenances allows impurities to excessively accumulate in spray water storage portion 17 that is short-term maintenance water storage portion 30. Setting predetermined time Tx to be equal to or less than twice time threshold value T1 can prevent degradation of the performance of the filter due to excessive accumulation of the impurities in spray water storage portion 17.

At time A in FIG. 5, next-maintenance notification determination unit 44 determines that the time obtained by adding predetermined time Tx to the long-term maintenance elapsed time is less than time threshold value T2. Therefore, the timing of the next short-term maintenance and the timing of the next long-term maintenance are not close to each other (S002: No), and thus next-maintenance notification determination unit 44 transmits, to maintenance notification unit 47, a statement that the notification of the short-term maintenance is to be performed in the next maintenance notification.

Then, short-term maintenance determination unit 45 determines whether or not the short-term maintenance elapsed time that is the elapsed time from completion of the previous short-term maintenance is equal to or more than time threshold value T1 (S007).

When the short-term maintenance elapsed time is equal to or more than time threshold value T1 (S007: Yes), short-term maintenance determination unit 45 determines that notification of maintenance is necessary, and transmits, to maintenance notification unit 47, the statement that notification is necessary. Maintenance notification unit 47 receives, from next-maintenance notification determination unit 44, the statement that the notification of the short-term maintenance is to be performed in the next maintenance notification, and further receives, from short-term maintenance determination unit 45, a determination that the notification is necessary, and thus maintenance notification unit 47 turns on the short-term maintenance LED in notification display 22 to perform the notification of the short-term maintenance (S010).

When the short-term maintenance elapsed time is less than time threshold value T1 (S007: No), short-term maintenance determination unit 45 determines whether or not the number of times of detection of water shortage counted by number counter 40 is equal to or more than number threshold value K1 (S009).

When the number of times of detection of water shortage is equal to or more than number threshold value K1 (S009: Yes), short-term maintenance determination unit 45 determines that notification of maintenance is necessary, and transmits, to maintenance notification unit 47, the statement that notification is necessary. Maintenance notification unit 47 receives, from next-maintenance notification determination unit 44, the statement that the notification of the short-term maintenance is to be performed in the next maintenance notification, and further receives, from short-term maintenance determination unit 45, a determination that the notification is necessary, and thus maintenance notification unit 47 turns on the short-term maintenance LED in notification display 22 to perform the notification of the short-term maintenance (S010).

When the number of times of detection of water shortage is less than number threshold value K1 (S009: No), short-term maintenance determination unit 45 determines that the maintenance notification is not necessary, and returns to the determination process of step S007 (S007).

At time B (timing B) in FIG. 5, the notification of the short-term maintenance is performed (S010), and the time from time A to time threshold value T1 or to when the number of times of detection of water shortage becomes equal to or more than number threshold value K1 has elapsed. In addition, when the short-term maintenance elapsed time is equal to or more than time threshold value T1, the notification of the short-term maintenance may be performed after the reception of the water shortage detection signal from detector 6. As a result, the short-term maintenance can be performed in a state where the amount of water in water storage portion 5 that is short-term maintenance water storage portion 30 is small, and thus it is possible to reduce the burden on the user carrying short-term maintenance water storage portion 30. In addition, spray water storage portion 17 may include a detector, and the notification of the short-term maintenance may be performed after the reception of the water shortage detection signal from the detector of spray water storage portion 17. As a result, the short-term maintenance can be performed in a state where the amount of water in short-term maintenance water storage portion 30 is small, and thus it is possible to reduce the burden on the user carrying short-term maintenance water storage portion 30.

At time C (timing C) in FIG. 5, a user completes the maintenance work and presses maintenance completion button 50, and then the statement that maintenance completion button 50 has been pressed is notified to controller 21. Maintenance notification unit 47 ends the notification of the short-term maintenance by turning off the short-term maintenance LED in notification display 22. As described above, impurities accumulated in short-term maintenance water storage portion 30 can be removed. Further, number counter 40 resets the number of times of detection of water shortage (S011). Thereafter, the process returns to step S002, and next-maintenance notification determination unit 44 determines that the time obtained by adding predetermined time Tx to the long-term maintenance elapsed time is less than time threshold value T2.

At time D (timing D) in FIG. 5, the notification of the short-term maintenance is performed similarly to time B. Thereafter, at times E, F, G, H, I, and J (timings E, F, G, H, I, and J), the same processing as the processing performed at time C and time D is repeated. That is, completion of the short-term maintenance and notification of the short-term maintenance are repeated. Thus, impurities accumulated in short-term maintenance water storage portion 30 can be removed periodically. In one example of the present exemplary embodiment with time threshold value T1 (30 days), number threshold value K1 (30), and time threshold value T2 (365 days), completion of the short-term maintenance and notification of the short-term maintenance are actually repeated a little more, but they are assumed to be repeated until time J for simplification of description.

The case where only the notification of the short-term maintenance is repeated has been described above. Hereinafter, a case where the notification of the short-term maintenance and the notification of the long-term maintenance are performed simultaneously in the next maintenance notification will be described.

At time K (timing K) in FIG. 5, the short-term maintenance is completed and then the process returns to step S002. Next-maintenance notification determination unit 44 determines that the time obtained by adding predetermined time Tx to the long-term maintenance elapsed time is equal to or more than time threshold value T2. Therefore, the timing of the next short-term maintenance and the timing of the next long-term maintenance are close to each other (S002: Yes), and thus next-maintenance notification determination unit 44 transmits, to maintenance notification unit 47, the statement that the notification of the short-term maintenance and the notification of the long-term maintenance are performed simultaneously in the next maintenance notification.

Then, long-term maintenance determination unit 46 determines whether or not the long-term maintenance elapsed time that is the elapsed time from completion of the previous long-term maintenance is equal to or more than time threshold value T2 (S003).

When the long-term maintenance elapsed time is equal to or longer than time threshold value T2 (S003: Yes), long-term maintenance determination unit 46 determines that notification of maintenance is necessary, and transmits, to maintenance notification unit 47, the statement that notification is necessary. Maintenance notification unit 47 receives, from next-maintenance notification determination unit 44, the statement that the notification of the short-term maintenance and the notification of the long-term maintenance are performed simultaneously in the next maintenance notification, and further receives, from long-term maintenance determination unit 46, a determination that the notification is necessary, and thus maintenance notification unit 47 simultaneously turns on the short-term maintenance LED and the long-term maintenance LED in notification display 22 to perform the notification of the short-term maintenance and the notification of the long-term maintenance simultaneously (S005).

When the long-term maintenance elapsed time is less than time threshold value T2 (S003: No), long-term maintenance determination unit 46 repeats the determination until the long-term maintenance elapsed time becomes equal to or more than time threshold value T2.

At time L (timing L) in FIG. 5, the notification of the short-term maintenance and the notification of the long-term maintenance are performed simultaneously (S005), and the time from time K to when the long-term maintenance elapsed time becomes equal to or more than time threshold value T2 has elapsed. In addition, when the long-term maintenance elapsed time is equal to or more than time threshold value T2, the notification of the short-term maintenance and the notification of the long-term maintenance may be performed simultaneously after the reception of the water shortage detection signal from detector 6. As a result, the short-term maintenance can be performed in a state where the amount of water in water storage portion 5 that is short-term maintenance water storage portion 30 is small, and thus it is possible to reduce the burden on the user carrying short-term maintenance water storage portion 30. In addition, spray water storage portion 17 may include a detector, and the notification of the short-term maintenance may be performed after the reception of the water shortage detection signal from the detector of spray water storage portion 17. As a result, the short-term maintenance can be performed in a state where the amount of water in short-term maintenance water storage portion 30 is small, and thus it is possible to reduce the burden on the user carrying short-term maintenance water storage portion 30.

At time M (timing M) in FIG. 5, the user completes the maintenance work and presses maintenance completion button 50, and then the statement that maintenance completion button 50 has been pressed is notified to controller 21. Maintenance notification unit 47 turns off the short-term maintenance LED and the long-term maintenance LED in notification display 22 to end the notification of the short-term maintenance and the notification of the long-term maintenance. Thus, impurities accumulated in short-term maintenance water storage portion 30 and long-term maintenance water storage portion 31 can be removed. Further, number counter 40 resets the number of times of detection, and time counter 41 resets the count value (S006).

With the above processing, when the timing of the long-term maintenance is approaching, the notification of the next short-term maintenance can be coincided with the timing of notification of the long-term maintenance. Simultaneously performing the notification of the short-term maintenance and the notification of the long-term maintenance can prevent, immediately after completion of one of the short-term maintenance and the long-term maintenance, the notification of the other maintenance from being performed. As a result, the user can perform both the short-term maintenance and the long-term maintenance upon a single notification, and can reduce the time and effort for maintenance. That is, the convenience of the user regarding the maintenance can be improved. In addition, a notification of only the short-term maintenance and simultaneous notification of the short-term maintenance and the long-term maintenance can be performed at appropriate timings.

### Second Exemplary Embodiment

In the first exemplary embodiment, when the notification of the short-term maintenance and the notification of the long-term maintenance are simultaneously performed, the notification of the short-term maintenance is coincided with the notification of the long-term maintenance. However, in second exemplary embodiment, when the notification of the short-term maintenance and the notification of the long-term maintenance are simultaneously performed, the notification of the long-term maintenance is coincided with the notification of the short-term maintenance. A configuration of electrolyzed water spraying device Z according to the second exemplary embodiment is similar to the configuration described with reference to FIGS. 1 and 2 in the first exemplary embodiment. Differences from the first exemplary embodiment will be mainly described.

A schematic functional block diagram of controller 21 and the peripheral units according to the second exemplary embodiment is substantially the same as the schematic functional block diagram in FIG. 3 described in the first exemplary embodiment, but long-term maintenance determination unit 46 need not be included. This is because the notification of the long-term maintenance is coincided with the notification of the short-term maintenance when the timing for the long-term maintenance is approaching. Detailed control contents will be described later.

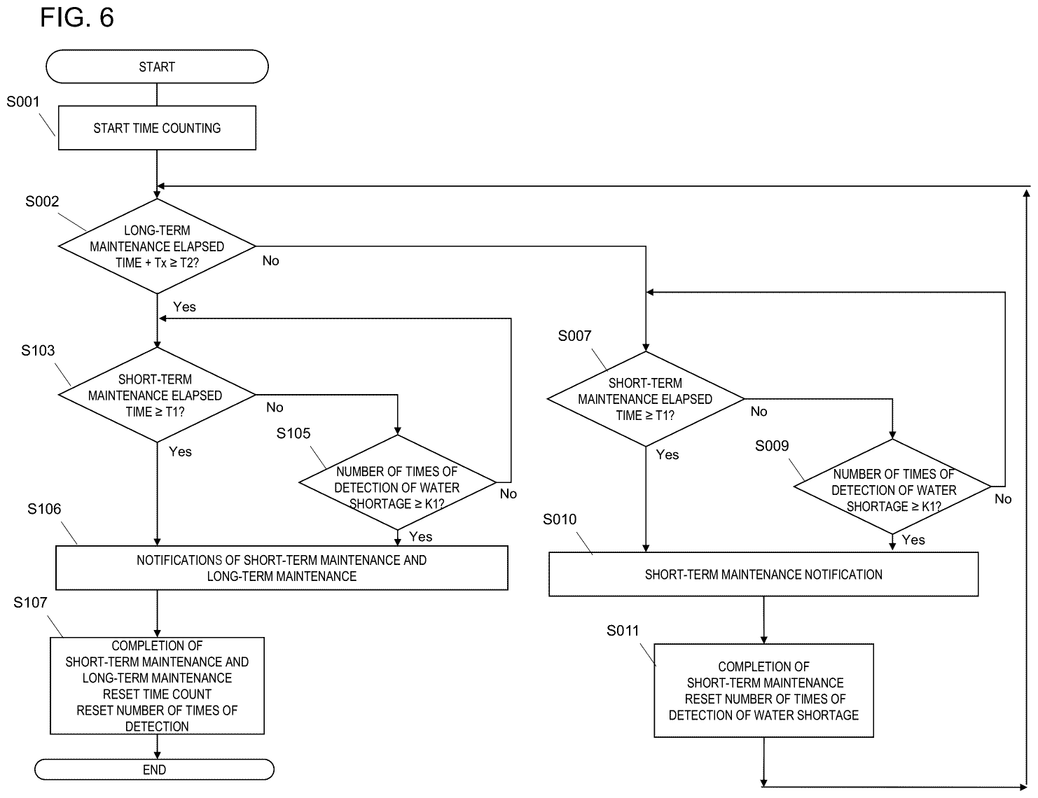

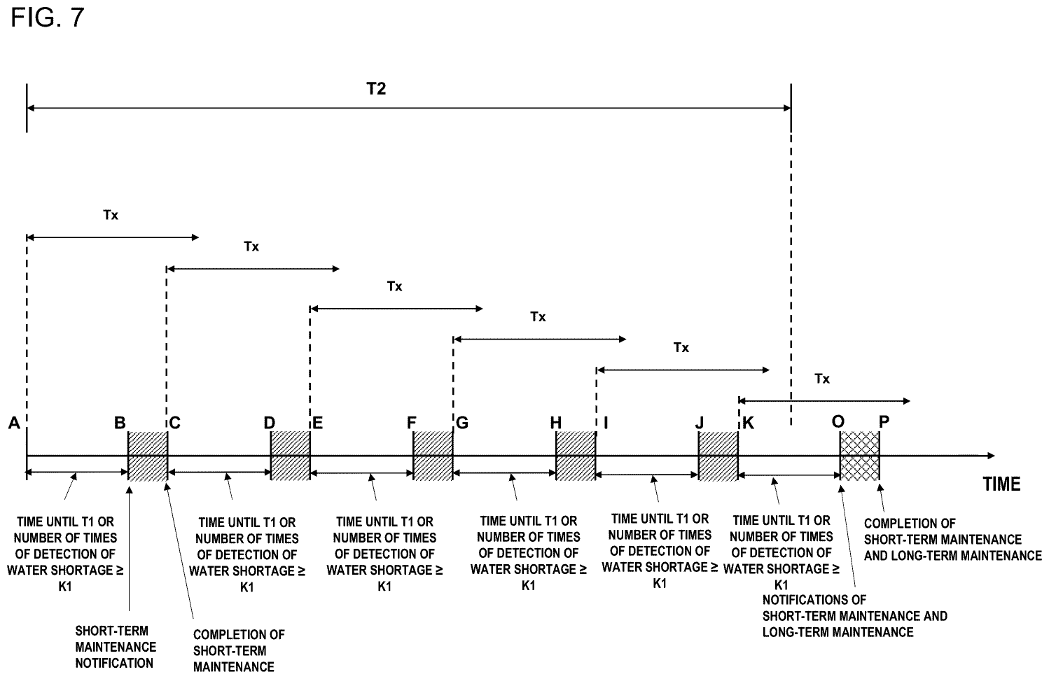

An operation of controller 21 with the above configuration will be described with reference to FIGS. 6 and 7. FIG. 6 is a flowchart illustrating a control procedure by controller 21. Herein, in the flowchart, numbers with S as an initial letter are assigned. For example, S001 or the like indicates a processing step. The magnitude of the numerical value indicating the processing step has no relation to the processing order. FIG. 7 is a diagram illustrating an example of the control procedure illustrated in FIG. 6 in time series.

In the second exemplary embodiment, the operation of controller 21 after completion of both the previous long-term maintenance and the short-term maintenance will be described. At times A, B, C, D, E, F, G, H, I, and J in FIG. 7, the same control as times A, B, C, D, E, F, G, H, I, and J in FIG. 5 of the first exemplary embodiment is performed, that is, the respective controls performed at steps S001, S002, S007, S009, S010, and S011 in FIG. 6 are the same as those at steps S001, S002, S007, S009, S010, and S011 in FIG. 4. Therefore, description up to time J is omitted.

At time K in FIG. 7, the short-term maintenance is completed and then the process returns to step S002. Next-maintenance notification determination unit 44 determines that the time obtained by adding predetermined time Tx to the long-term maintenance elapsed time is equal to or more than time threshold value T2. Therefore, the timing of the next short-term maintenance and the timing of the next long-term maintenance are close to each other (S002: Yes), and thus next-maintenance notification determination unit 44 transmits, to maintenance notification unit 47, the statement that the notification of the short-term maintenance and the notification of the long-term maintenance are performed simultaneously in the next maintenance notification.

Then, short-term maintenance determination unit 45 determines whether or not the short-term maintenance elapsed time that is the elapsed time from completion of the previous short-term maintenance is equal to or more than time threshold value T1 (S103).

When the short-term maintenance elapsed time is equal to or more than time threshold value T1 (S103: Yes), short-term maintenance determination unit 45 determines that notification of maintenance is necessary, and transmits, to maintenance notification unit 47, the statement that notification is necessary. Maintenance notification unit 47 receives, from next-maintenance notification determination unit 44, the statement that the notification of the short-term maintenance and the notification of the long-term maintenance are performed simultaneously in the next maintenance notification, and further receives, from short-term maintenance determination unit 45, a determination that the notification is necessary, and thus maintenance notification unit 47 simultaneously turns on the short-term maintenance LED and the long-term maintenance LED in notification display 22 to perform the notification of the short-term maintenance and the notification of the long-term maintenance simultaneously (S106).

When the short-term maintenance elapsed time is less than time threshold value T1 (S103: No), short-term maintenance determination unit 45 determines whether or not the number of times of detection of water shortage counted by number counter 40 is equal to or more than number threshold value K1 (S105).

When the number of times of detection of water shortage is equal to or more than number threshold value K1 (S105: Yes), short-term maintenance determination unit 45 determines that notification of maintenance is necessary, and transmits, to maintenance notification unit 47, the statement that notification is necessary. Maintenance notification unit 47 receives, from next-maintenance notification determination unit 44, the statement that the notification of the short-term maintenance and the notification of the long-term maintenance are performed simultaneously in the next maintenance notification, and further receives, from short-term maintenance determination unit 45, a determination that the notification is necessary, and thus maintenance notification unit 47 simultaneously turns on the short-term maintenance LED and the long-term maintenance LED in notification display 22 to perform the notification of the short-term maintenance and the notification of the long-term maintenance simultaneously (S106).

When the number of times of detection of water shortage is less than number threshold value K1 (S105: No), short-term maintenance determination unit 45 determines that the maintenance notification is not necessary, and returns to the determination process of step S103.

At time O (timing O) in FIG. 7, the notification of the short-term maintenance and the notification of the long-term maintenance are performed simultaneously (S106), and the time from time K to time threshold value T1 or to when the number of times of detection of water shortage reaches a number equal to or more than number threshold value K1 has elapsed. In addition, when the short-term maintenance elapsed time is equal to or more than time threshold value T1 in step S103, the notification of the short-term maintenance and the notification of the long-term maintenance may be performed simultaneously after the reception of the water shortage detection signal from detector 6. As a result, the short-term maintenance can be performed in a state where the amount of water in water storage portion 5 that is short-term maintenance water storage portion 30 is small, and thus it is possible to reduce the burden on the user carrying short-term maintenance water storage portion 30. In addition, spray water storage portion 17 may include a detector, and the notification of the short-term maintenance may be performed after the reception of the water shortage detection signal from the detector of spray water storage portion 17. As a result, the short-term maintenance can be performed in a state where the amount of water in short-term maintenance water storage portion 30 is small, and thus it is possible to reduce the burden on the user carrying short-term maintenance water storage portion 30.

At time P (timing P) in FIG. 7, the user completes the maintenance work and presses maintenance completion button 50, and then the statement that maintenance completion button 50 has been pressed is notified to controller 21. Maintenance notification unit 47 turns off the short-term maintenance LED and the long-term maintenance LED in notification display 22 to end the notification of the short-term maintenance and the notification of the long-term maintenance. Thus, impurities accumulated in short-term maintenance water storage portion 30 and long-term maintenance water storage portion 31 can be removed. Further, number counter 40 resets the number of times of detection, and time counter 41 resets the count value (S107).

With the above processing, when the timing of the long-term maintenance is approaching, the notification of the next long-term maintenance can be coincided with the timing of notification of the short-term maintenance. Simultaneously performing the notification of the short-term maintenance and the notification of the long-term maintenance can prevent, immediately after completion of one of the short-term maintenance and the long-term maintenance, the notification of the other maintenance from being performed. As a result, the user can perform both the short-term maintenance and the long-term maintenance upon a single notification, and can reduce the time and effort for maintenance. That is, the convenience of the user regarding the maintenance can be improved. In addition, a notification of only the short-term maintenance and simultaneous notification of the short-term maintenance and the long-term maintenance can be performed at appropriate timings.

The present disclosure has been described above based on the exemplary embodiment. It will be understood by those who are skilled in the art that the present exemplary embodiment is merely an example, that various modifications of combinations of components and/or processes of the exemplary embodiment are possible, and that such modifications still fall within the scope of the present disclosure.

For example, controller 21 (notification controller 43) may further include control content memory unit 48 that stores control content currently being executed. An example of control content memory unit 48 is a nonvolatile memory. Controller 21 periodically stores the control content currently being executed in control content memory unit 48 as necessary. When the power supply of electrolyzed water spraying device Z is shut off and then the power supply is restored, controller 21 resumes from the control content that was being executed and stored in control content memory unit 48. As a result, even when the power supply of electrolyzed water spraying device Z is shut off and then the power supply is restored, correct control contents can be performed.

An electrolyzed water spraying device according to the present disclosure is an electrolyzed water spraying device that discharges electrolyzed water, and includes: a short-term maintenance water storage portion that stores liquid and requires short-term maintenance that is maintenance on a short-term basis; a long-term maintenance water storage portion that stores the liquid and requires long-term maintenance that is maintenance on a long-term basis longer than the maintenance of the short-term maintenance water storage portion; and a notification controller that notifies each of a timing of a next short-term maintenance of the short-term maintenance water storage portion and a timing of a next long-term maintenance of the long-term maintenance water storage portion. The notification controller simultaneously performs the notification of the timing of the next short-term maintenance and the notification of the timing of the next long-term maintenance when the timing of the next short-term maintenance and the timing of the next long-term maintenance are close to each other.

As a result, immediately after one of the short-term maintenance and the long-term maintenance, the notification of the other maintenance is not performed, so that the time and effort for maintenance by the user can be reduced. That is, the convenience of the user regarding the maintenance can be improved.

In addition, the notification of the next short-term maintenance may be determined based on at least one of the number of times of water shortage in the short-term maintenance water storage portion or the number of times of water supply maintenance that is supply of liquid to the short-term maintenance water storage portion and the elapsed time from the previous short-term maintenance for the short-term maintenance water storage portion, or the elapsed time from the previous long-term maintenance for the long-term maintenance water storage portion. The notification of the next long-term maintenance may be determined based on at least one of the elapsed time from the previous long-term maintenance for the long-term maintenance water storage portion, the number of times of water shortage in the short-term maintenance water storage portion, or the number of times of water supply maintenance of the short-term maintenance water storage portion, and the elapsed time from the previous short-term maintenance for the short-term maintenance water storage portion. As a result, the notification of the short-term maintenance and the notification of the long-term maintenance can be performed at appropriate timings.

In addition, the notification of the next short-term maintenance may be determined based on both the number of times of water shortage in the short-term maintenance water storage portion or the number of times of water supply maintenance that is supply of liquid to the short-term maintenance water storage portion, and the elapsed time from the previous short-term maintenance for the short-term maintenance water storage portion. As a result, the notification of the short-term maintenance and the notification of the long-term maintenance can be performed at appropriate timings.

The short-term maintenance may also include at least one of draining or cleaning and water supply maintenance performed after at least one of the draining or the cleaning is performed. The long-term maintenance may include at least one of the draining or the cleaning. As a result, the short-term maintenance water storage portion and the long-term maintenance water storage portion can be maintained clean. In addition, impurities accumulated in short-term maintenance water storage portion and long-term maintenance water storage portion can be removed.

The electrolyzed water spraying device may further include: a detector that detects water shortage of the liquid in the short-term maintenance water storage portion or supply of the liquid to the short-term maintenance water storage portion; a number counter that counts the number of times of the detection by the detector as the number of times of detection; a time counter that counts an elapsed time from completion of the previous long-term maintenance of the long-term maintenance water storage portion; and a memory unit that stores time threshold value T1 used for determination of the notification of the next short-term maintenance or number threshold value K1 of the detection, and time threshold value T2 larger than time threshold value T1 and used for determination of the notification of the next long-term maintenance. In a case where a time obtained by adding predetermined time Tx to a count value counted by the time counter at the time of completion of the previous short-term maintenance is equal to or less than time threshold value T2, the notification controller may perform the notification of the next short-term maintenance when the elapsed time from completion of the previous short-term maintenance is equal to or more than time threshold value T1 or the number of times of detection from completion of the previous short-term maintenance is equal to or more than number threshold value K1. In a case where the time obtained by adding predetermined time Tx to the count value counted by the time counter at the time of completion of the previous short-term maintenance is longer than time threshold value T2, the notification controller may simultaneously perform the notification of the next short-term maintenance and the notification of the next long-term maintenance. As a result, a notification of only the short-term maintenance and simultaneous notification of the short-term maintenance and the long-term maintenance can be performed at appropriate timings.

In addition, when the time obtained by adding predetermined time Tx to the count value counted by the time counter at the time of completion of the previous short-term maintenance is longer than time threshold value T2, the notification controller may simultaneously perform the notification of the next short-term maintenance and the notification of the next long-term maintenance when the count value is equal to or more than time threshold value T2 in order to coincide the notification of the next short-term maintenance with the notification of the next long-term maintenance. As a result, the notification of the short-term maintenance can be coincided with the notification of the long-term maintenance, and it is possible to prevent the notification of the short-term maintenance from being performed near the time of the notification of the long-term maintenance.

In addition, in a case where the time obtained by adding predetermined time Tx to the count value counted by the time counter at the time of completion of the previous short-term maintenance is longer than time threshold value T2, the notification controller may simultaneously perform the notification of the next short-term maintenance and the notification of the next long-term maintenance when the elapsed time from completion of the previous short-term maintenance is equal to or more than time threshold value T1 or the number of times of detection from completion of the previous short-term maintenance is equal to or more than number threshold value K1 in order to coincide the notification of the next long-term maintenance with the notification of the next short-term maintenance. As a result, the notification of the long-term maintenance can be coincided with the notification of the short-term maintenance, and it is possible to prevent the notification of the long-term maintenance from being performed near the time of notification of the short-term maintenance.

In addition, predetermined time Tx may be equal to or less than twice the value of time threshold value T1. This can prevent impurities from excessively accumulating in the short-term maintenance water storage portion.

In addition, the detector may detect water shortage in the short-term maintenance water storage portion. In a case where the time obtained by adding predetermined time Tx to the count value counted by the time counter at the time of completion of the previous short-term maintenance is equal to or less than time threshold value T2, the notification controller may perform the notification of the next short-term maintenance after the detector detects the water shortage in the short-term maintenance water storage portion when the elapsed time from completion of the previous short-term maintenance is equal to or more than time threshold value T1. As a result, when the short-term maintenance water storage portion is in the water shortage state, the notification of the short-term maintenance can be performed, and it is possible to reduce the burden on the user carrying the short-term maintenance water storage portion.

In addition, the detector detects the water shortage in the short-term maintenance water storage portion, and the notification controller may perform the notification after the detector detects the water shortage in the short-term maintenance water storage portion when simultaneously performing the notification of the next short-term maintenance and the notification of the next long-term maintenance. As a result, when the short-term maintenance water storage portion is in the water shortage state, the notification of the short-term maintenance and the notification of the long-term maintenance can be simultaneously performed, and it is possible to reduce the burden on the user carrying the short-term maintenance water storage portion.

In addition, the long-term maintenance water storage portion may be an electrolyzed water storage portion that stores the electrolyzed water, and the short-term maintenance water storage portion may be a water storage portion that stores the water, and a spray water storage portion that stores the spray water obtained by mixing the water in the water storage portion and the electrolyzed water in the electrolyzed water storage portion. As a result, maintenance of the electrolyzed water storage portion, the water storage portion, and the spray water storage portion can be performed at appropriate timings.

### INDUSTRIAL APPLICABILITY

The electrolyzed water spraying device according to the present disclosure is useful as an electrolyzed water spraying device that removes (including inactivation of) bacteria, fungi, viruses, odors, and the like in the air.

### REFERENCE MARKS IN THE DRAWINGS

Z electrolyzed water spraying device
1 body case
2 air inlet
3 panel
4 air outlet
5 water storage portion
6 detector
7 first pump
8 second pump
9 first water conveyance path
10 second water conveyance path
11 water supply portion
12 electrolyzed water storage portion
13 electrolytic accelerator charge portion
14 electrolysis unit
15 third pump
16 third water conveyance path
17 spray water storage portion
18 spray portion
19 air blower
20 air passage
21 controller
22 notification display
23 operation portion
24 supply port
30 short-term maintenance water storage portion
31 long-term maintenance water storage portion
40 number counter
41 time counter
42 memory unit
43 notification controller
44 next-maintenance notification determination unit
45 short-term maintenance determination unit
46 long-term maintenance determination unit
47 maintenance notification unit
48 control content memory unit
50 maintenance completion button

What is claimed is:

1. An electrolyzed water spraying device that discharges electrolyzed water, comprising:
a short-term maintenance water storage portion that stores liquid and requires short-term maintenance that is maintenance on a short-term basis;
a long-term maintenance water storage portion that stores the liquid and requires long-term maintenance that is maintenance on a long-term basis longer than the maintenance of the short-term maintenance water storage portion; and
a notification controller that notifies each of a timing of a next short-term maintenance of the short-term maintenance water storage portion and a timing of a next long-term maintenance of the long-term maintenance water storage portion,
wherein the notification controller simultaneously performs a notification of the timing of the next short-term maintenance and a notification of the timing of the next long-term maintenance when the timing of the next short-term maintenance and the timing of the next long-term maintenance are close to each other.

2. The electrolyzed water spraying device according to claim 1, wherein
the notification of the next short-term maintenance is determined based on at least one of a number of times of water shortage in the short-term maintenance water storage portion or a number of times of water supply maintenance that is supply of the liquid to the short-term maintenance water storage portion, and an elapsed time from previous short-term maintenance for the short-term maintenance water storage portion, or an elapsed time from previous long-term maintenance for the long-term maintenance water storage portion, and
the notification of the next long-term maintenance is determined based on the elapsed time from the previous long-term maintenance for the long-term maintenance water storage portion, or at least one of the number of times of water shortage in the short-term maintenance water storage portion or the number of times of water supply maintenance of the short-term maintenance water storage portion, and the elapsed time from the previous short-term maintenance for the short-term maintenance water storage portion.

3. The electrolyzed water spraying device according to claim 2, wherein
the notification of the next short-term maintenance is determined based on both the number of times of water shortage in the short-term maintenance water storage portion or the number of times of the water supply maintenance that is supply of the liquid to the short-term maintenance water storage portion and the elapsed time from the previous short-term maintenance for the short-term maintenance water storage portion.

4. The electrolyzed water spraying device according to claim 2, wherein
the short-term maintenance includes:
at least one of draining or cleaning; and
the water supply maintenance performed after at least one of the draining or the cleaning, and
the long-term maintenance includes at least one of the draining or the cleaning.

5. The electrolyzed water spraying device according to claim 2, further comprising:
a detector that detects water shortage of the liquid in the short-term maintenance water storage portion or supply of the liquid to the short-term maintenance water storage portion;
a number counter that counts a number of times of the detection by the detector as a number of times of detection;
a time counter that counts an elapsed time from completion of the previous long-term maintenance of the long-term maintenance water storage portion; and
a memory unit that stores time threshold value T1 used for determination of a notification of the next short-term maintenance or number threshold value K1 of the detection, and time threshold value T2 larger than the time threshold value T1 and used for determination of a notification of the next long-term maintenance,
wherein
in a case where a time obtained by adding predetermined time Tx to a count value counted by the time counter at time of completion of the previous short-term maintenance is equal to or less than the time threshold value T2,
the notification controller performs the notification of the next short-term maintenance when the elapsed time from the completion of the previous short-term maintenance is equal to or more than the time threshold value T1 or the number of times of the detection from the completion of the previous short-term maintenance is equal to or more than the number threshold value K1, and
in a case where the time obtained by adding predetermined time Tx to the count value counted by the time counter at time of the completion of the previous short-term maintenance is longer than the time threshold value T2,
the notification controller simultaneously performs the notification of the next short-term maintenance and the notification of the next long-term maintenance.

6. The electrolyzed water spraying device according to claim 5, wherein
in a case where the time obtained by adding predetermined time Tx to the count value counted by the time counter at time of the completion of the previous short-term maintenance is longer than the time threshold value T2,
in order to coincide the notification of the next short-term maintenance with the notification of next the long-term maintenance, the notification controller performs the notification of the next short-term maintenance and the notification of the next long-term maintenance simultaneously when the count value is equal to or more than the time threshold value T2.

7. The electrolyzed water spraying device according to claim 5, wherein
in a case where the time obtained by adding the predetermined time Tx to the count value counted by the time counter at time of the completion of the previous short-term maintenance is longer than the time threshold value T2,
in order to coincide the notification of the next long-term maintenance with the notification of the next short-term maintenance, the notification controller performs the notification of the next short-term maintenance and the notification of the next long-term maintenance simultaneously when the elapsed time from the completion of the previous short-term maintenance is equal to or more than the time threshold value T1, or the number of times of detection from the completion of the previous short-term maintenance is equal to or more than the number threshold value K1.

8. The electrolyzed water spraying device according to claim 5, wherein the predetermined time Tx is equal to or less than twice the time threshold value T1.

9. The electrolyzed water spraying device according to claim 5, wherein
the detector detects water shortage of the liquid in the short-term maintenance water storage portion, and
in a case where the time obtained by adding predetermined time Tx to the count value counted by the time counter at time of the completion of the previous short-term maintenance is equal to or less than the time threshold value T2,
the notification controller performs the notification of the next short-term maintenance after the detector detects water shortage in the short-term maintenance water storage portion when the elapsed time from the completion of the previous short-term maintenance is equal to or more than the time threshold value T1.

10. The electrolyzed water spraying device according to claim 6, wherein
the detector detects water shortage of the liquid in the short-term maintenance water storage portion, and
the notification controller performs the notification after the detector detects water shortage in the short-term maintenance water storage portion when the notification of the next short-term maintenance and the notification of the next long-term maintenance are simultaneously performed.

11. The electrolyzed water spraying device according to claim 1, wherein
the long-term maintenance water storage portion is an electrolyzed water storage portion that stores the electrolyzed water, and
the short-term maintenance water storage portion is a water storage portion that stores water, and a spray water storage portion that stores spray water obtained by mixing the water in the water storage portion and the electrolyzed water in the electrolyzed water storage portion.

12. The electrolyzed water spraying device according to claim 1, further comprising a control content memory unit that stores a control content currently being executed,
wherein when power supply is shut off and then the power supply is restored, the notification controller resumes from the control content that was being executed and stored in the control content memory unit.

13. The electrolyzed water spraying device according to claim 7, wherein
the detector detects water shortage of the liquid in the short-term maintenance water storage portion, and
the notification controller performs the notification after the detector detects water shortage in the short-term maintenance water storage portion when the notification of the next short-term maintenance and the notification of the next long-term maintenance are simultaneously performed.
